(19) United States
(12) Patent Application Publication
(10) Pub. No.: US 2007/0267585 A1
(43) Pub. Date: Nov. 22, 2007

(54) METHOD AND SYSTEM FOR DIGITAL INTRAORAL X-RAY OF NON-HUMAN ANIMALS

(76) Inventors: Scott T. Porter, Granada Hills, CA (US); Charles D. Emig, Maple Valley, WA (US); Philip E. Goeschl, Northridge, CA (US); Richard L. Vetter, Chehalis, WA (US); Andrea Becker, Federal Way, WA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/633,195

(22) Filed: Dec. 4, 2006

Related U.S. Application Data
(60) Provisional application No. 60/741,706, filed on Dec. 2, 2005.

(51) Int. Cl. G03B 42/08; A61B 6/14

(52) U.S. Cl. 250/583; 378/169

(57) ABSTRACT

Embodiments include a method and system for digital intraoral x-ray of non-human animals using a flexible and reusable cassette and imaging plate. The imaging plate is activated using an x-ray generator and scanned to capture an image from the imaging plate.

### CROSS REFERENCE TO RELATED APPLICATIONS

This application claims priority from U.S. Provisional Application Ser. No. 60/741,706, filed on Dec. 2, 2005, which is incorporated herein by reference in its entirety.

### BACKGROUND

1. Field of the Invention

The embodiments of the invention relate generally to digital intraoral x-ray imaging of non-human animals. Specifically, embodiments of the invention relate to digital intraoral x-ray imaging of equines using flexible and reusable x-ray cassettes and imaging plates.

2. Background

Animals, much like humans, suffer from periodontal diseases and other dental issues. Accordingly, dental techniques, such as radiography using x-ray film, have been used on animals for decades. In the case of equines, typically the film is placed on one side of the head and an x-ray source is placed on the other side. The result is a radiograph that shows both the near and far set of teeth superimposed on one another.

Over the last three to five years equine dentistry has evolved to include the use of x-ray film in a cassette that protects the film from ambient light. Animals such as equines, however, typically must be drugged during the procedure to prevent them from biting, chewing, etc. during the procedure. Since the animal can only be safely held in a drugged state for a short period of time, the procedure must be performed relatively quickly. This often poses a problem because multiple images are typically necessary to diagnose soft tissue problems as well as tooth and pulp issues. To develop the film, the film is transferred to a dark room and exposed using various chemicals. In this aspect, it is often difficult to develop the film on site since most locations where animals are kept, for example barns, do not have a dark room or the necessary chemicals.

To overcome some of these limitations, attempts have been made to use digital radiology (DR) systems which do not require a dark room. DR provides an x-ray system which uses sensors, such as charge coupled device (CCD) sensors, placed inside the mouth. The result is an immediate digital image which does not need to be developed in a dark room. DR, however, although practical for use with humans, does not provide a practical solution in the case of animals. In particular, DR sensors are delicate and can be expensive to repair or replace. In addition, a cord must be connected from the DR sensor to electronically transmit the digital image to a computer. Non-human animal behavior, however, is highly unpredictable and difficult to control. Accordingly, placing a delicate and expensive component such as a DR sensor within the jaws of an animal, such as an equine, is likely to result in irreparable damage to the sensor. Thus, making DR undesirable for use with non-human animals.

Although recently, the dental profession has begun to use Computed Radiography (CR) on humans, this technique is not typically used for equine. CR involves the use of plates to store a latent image in a photostimulable storage phosphor plate. The plate is reflective and erasable allowing repeated exposures; the number of possible exposures is greater than 150,000 if there are no mechanical stresses on the material. An x-ray impregnates the plate with energy which, when subsequently exposed to a particular excitation, is released, recreating the image. Scanners or CR readers of these plates typically include a flying spot laser and photomultiplier tube sensor. The photo multiplier tube captures the image corresponding to the energy being released by excitation of the flying spot laser. The image can then be viewed and in some cases enhanced using specialized software on a computing device.

### BRIEF DESCRIPTION OF THE DRAWINGS

Embodiments of the invention are illustrated by way of example and not by way of limitation in the figures of the accompanying drawings in which like references indicate similar elements. It should be noted that different references to “an” or “one” embodiment in this disclosure are not necessarily to the same embodiment, and such references mean at least one.

FIG. 1 is a diagram of a carrier positioned within an oral region of a non-human animal.

FIG. 2 is an image obtained from an imaging plate within the carrier of FIG. 1.

FIG. 3 is diagram of an open carrier with an imaging plate disposed therein.

FIG. 4 is a diagram of a closed carrier.

FIG. 5 is a diagram of a closed carrier and holder.

FIG. 6 is a diagram of a carrier positioned within an oral region of a non-human animal using a holder.

FIG. 7 is an image obtained from an imaging plate within the carrier of FIG. 6.

FIG. 8 is a diagram of an imaging plate positioned within a processing cassette.

FIG. 9 is a diagram of a scanner with a processing cassette and imaging plate positioned therein.

FIG. 10 is a flow chart of an imaging operation of a system for intraoral imaging.

FIG. 11 is a flow chart of a processing operation of a system for intraoral imaging.

### DETAILED DESCRIPTION

The methods and systems described herein provide reusable components for obtaining intraoral images of non-human animals on site (e.g. in a barn) or at a medical facility.

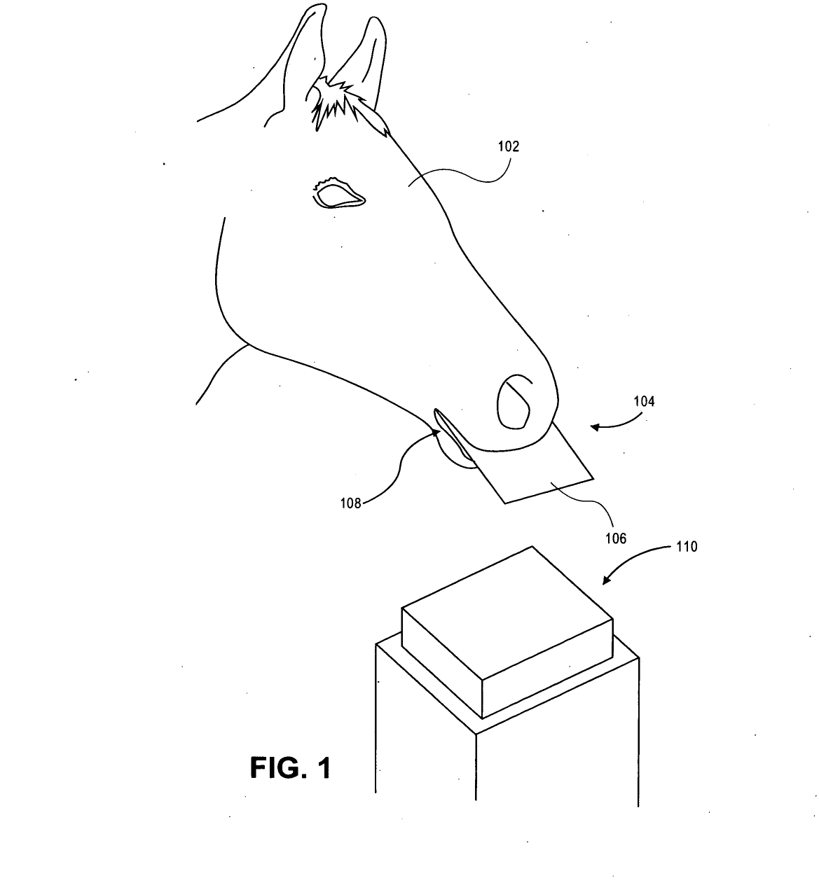

FIG. 1 is a diagram showing a carrier positioned within an oral region, for example a mouth, of a non-human animal. In this embodiment, the non-human animal is an equine 102. It is further contemplated that the non-human animal may be any non-human member of the kingdom Animalia deemed desirable, for example, large animals such as giraffes, hippopotamuses, whales and dolphins; large dogs; and farm animals such as sheep, goats and bovine.

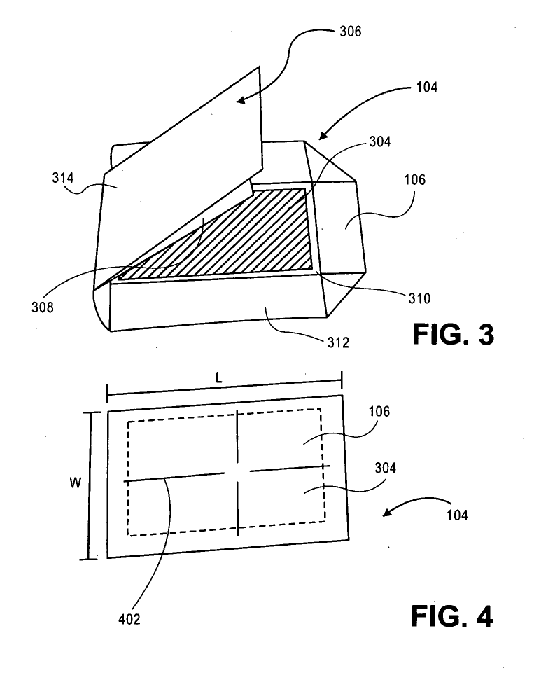

Cassette or carrier 104 having an imaging plate 302 (See FIG. 3) diposed therein is shown within oral region 108 of equine 102. Carrier 104 may include housing 106 dimensioned to fit within oral region 108 and hold imaging plate 304. Housing 106 may be made of a flexible material suitable to withstand the wear and tear it may experience when used within an oral region of a non-human animal. Carrier 104 may further be reusable therefore suitable materials for housing 106 may be those which can be sterilized or otherwise cleaned, for example with alcohol, without affecting the performance of carrier 104. In some embodiments, a suitable material for housing 106 may be a vinyl or other similar materials.

In this embodiment, carrier 104 is positioned horizontally between an upper and lower jaw of equine 102. X-ray generator 110 is positioned below the head of equine 102 and in turn below carrier 104. In this embodiment, x-ray generator 110 may be used to activate photostimulable material on imaging plate 302 so that tissues and structures within a lower portion of oral region 108, for example a lower jaw and teeth, may be imaged.

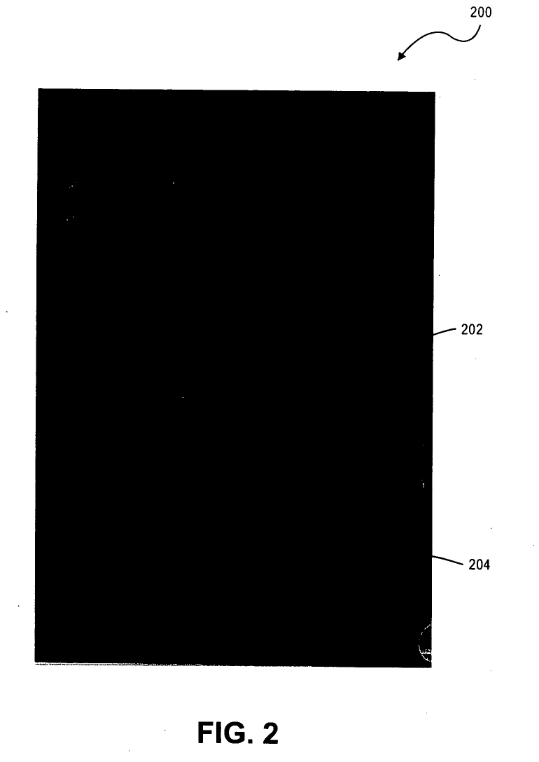

FIG. 2 shows an exemplary radiograph image 200 that may be obtained when carrier 104 with imaging plate 304 inside is positioned horizontally between teeth of equine 102 as shown in FIG. 1. In this aspect, an isolated portion of oral region 108 including lower jaw 204, teeth 202 extending from lower jaw 204 and associated tissues may be imaged and examined by a dentist. Since only teeth 202 along lower jaw 204 are imaged, as opposed to a superimposed image of both upper and lower jaw teeth sets, the dentist is able to identify each tooth and examine each one individually. Alternatively, the horizontally positioned imaging plate 304 as shown may be used to image the upper jaw and associated structures and tissues by turning carrier 104 over and positioning x-ray generator 110 above oral region 108 such that radiation is directed from the top down.

FIG. 3 is diagram of an open reusable carrier with an imaging plated disposed therein. Carrier 104 includes housing 106 and imaging plate 304 positioned within housing 106. In this embodiment, housing 106 of carrier 104 is shown having a plurality of flaps 306. In this aspect, carrier 104 may be opened and closed by folding or unfolding flaps 306. Imaging plate 304 may be retained within carrier 104 by inserting imaging plate 304 within housing 106 when in the open configuration and folding flaps 306 around it. For example, in the embodiment of FIG. 3, imaging plate 304 is positioned on base member 310 of housing 106 which is exposed when flaps 306 are unfolded as shown. Second flap 308 is then folded over peripheral flaps 312. Peripheral flaps 312 extending from base member 210 are then folded over second flap 308. Finally, cover 314 is folded down over peripheral flaps 312 to completely enclose imaging plate 304 within housing 106. In this aspect, housing 106 protects imaging plate 304 from ambient energy (light) sources that could affect imaging plate 304. To remove imaging plate 304, flaps 306 are unfolded as shown in FIG. 3. Alternatively, carrier 104 may have any configuration that is reusable and suitable for protecting and removably retaining imaging plate 304 positioned therein.

Imaging plate 304 may be a photostimulable plate covered in a phosphor storage material used to capture x-ray images. For example, imaging plate 304 may be a phosphorescent plate. In another embodiment, other photostimulable materials may be used to construct imaging plate 304.

FIG. 4 is a diagram of a closed carrier. In this embodiment, imaging plate 304 is shown positioned within housing 106. Although carrier 104 is shown having a substantially rectangular shape, it is contemplated that housing 106 may have any shape and be of any dimension suitable for holding imaging plate 304 therein and positioning carrier 104 within an oral region, for example a mouth, of a non-human animal. In the case of a rectangular shape, a suitable dimension may be any dimension having a width (W) greater than 2¼ inches and a length (L) greater than 3 inches. In some embodiments, housing 106 may have a length of 7 inches and a width of 3½ inches, still further housing 106 may have a length of 8½ inches and a width of 4½ inches. In turn, imaging plate 304 may have a shape and dimension suitable to fit within housing 106. It is further contemplated that the size and dimensions of housing 106 of carrier 104 may be modified as desired depending upon the size of the non-human animal and in turn oral region in which it is to be used. For example, in some embodiments, carrier 104 may have a square, circular or elliptical shape and imaging plate 304 may have the same or different shape.

In some embodiments, label 402 may be included on an outer surface of housing 106. Label 402 may be used to identify which side of carrier 104 is to face x-ray generator 110. FIG. 4 illustrates a label in the form of a target symbol. It is further contemplated that additional labels may be used along an outer surface of housing 106 to identify characteristics such as a size of carrier 104.

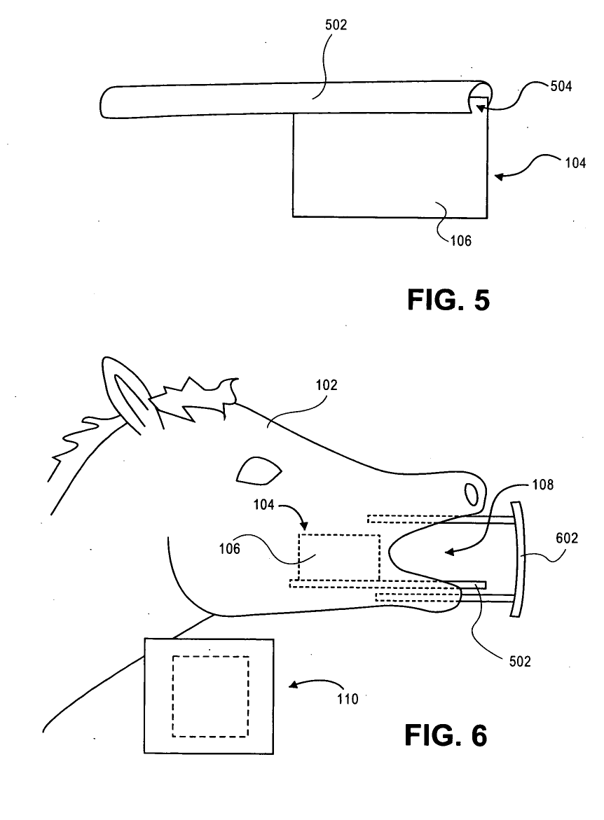

FIG. 5 is a diagram of a closed carrier and holder. Holder 502 may be connected to carrier 104 and used to insert and position carrier 104 within an oral region of, for example, equine 102. In this embodiment, holder 502 is a flexible tubular structure connected at one end to an edge of carrier 104. In this aspect, holder 502 includes a slot along a portion of its length which is wide enough to receive and clamp onto an edge of housing 106 of carrier 104. In some embodiments, holder 502 may be formed by cutting a slot along a length of a polyvinyl chloride (PVC) pipe. In some embodiments, a suitable circumference of tubular holder 502 may be at least ½ inch and still further from about ¼ inch to about ⅜ inch. Holder 502 may have any length suitable for holding carrier 104 at one end while simultaneously allowing a user to comfortably grasp an opposite end. For example, in some embodiments, holder 502 may have a length of about 12 inches to 18 inches. It is further contemplated that holder 502 may have any shape and size and be made of any material suitable for facilitating positioning of carrier 104 within an oral region. It is desirable to use a holder that will not negatively impact the capture of an x-ray image on imaging plate 304 within carrier 104. Thus, metal holders are deemed less desirable than non-metal holders. It is further contemplated that holder 502 may be reused thus in some embodiments, holder 502 may be of a material capable of being cleaned between uses without compromising the functionality of holder 502.

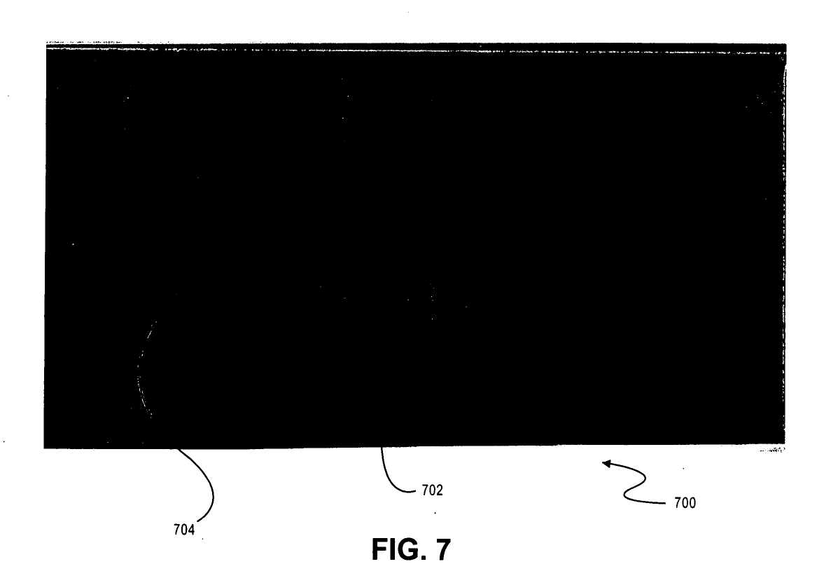

FIG. 6 is a diagram of a reusable cassette positioned within an oral region of a non-human animal using a holder and expansion mechanism. In this embodiment, carrier 104 with imaging plate 304 inside is shown positioned along a side of oral region 108 of equine 102. For example, carrier 104 may be positioned between a tongue and teeth of equine 102. X-ray generator 110 may then be positioned along a side of equine 102 such that it is substantially perpendicular and in-line with carrier 104. Positioning carrier 104 and x-ray generator 110 in this manner allows a user to obtain an image of, for example, teeth 702 from an upper jaw 704 as shown in radiograph image 700 of FIG. 7. In addition, the shape and size of carrier 104 and corresponding imaging plate 304 as described herein, allows for imaging of, in addition to the teeth and upper jaw, surrounding tissue, sinus cavities and other anatomical features. It is further contemplated that carrier 104 may be positioned differently within oral region 108 and x-ray generator 110 may be positioned differently around the head of equine 102 to image another portion of oral region 108.

In this embodiment, carrier 104 may be positioned deeper in oral region 108 than when carrier 104 is positioned between the teeth of equine 102 as illustrated in FIG. 1. In this aspect, holder 502 may be used to help position carrier 104 within oral region 108 to facilitate imaging of, in addition to the teeth and upper jaw, surrounding tissue, sinus cavities and other anatomical features. Additionally, expansion mechanism 602 may be used to expand an opening of oral region 108 to facilitate insertion of carrier 104 within oral region 108. In some embodiments, expansion mechanism 602 may be a speculum such as that commercially available from Capps Manufacturing Inc., of Caltonia, Nebr., under the model number WWE2000. As illustrated in FIG. 6, speculum 602 is inserted into oral region 108 along a gumline of equine 102 and expanded to dilate and maintain an opening to oral region 108. Alternatively, any type of expansion mechanism 602 suitable for increasing and maintaining an opening to oral region 108 may be used.

Once imaging plate 304 within carrier 104 is stimulated by, for example, x-rays emitted from x-ray generator 110, carrier 104 may be removed from oral region 108 and imaging plate 304 may be removed from carrier 104 and placed within a processing cassette for scanning. Care, however, must be taken when transferring imaging plate 304 from carrier 104 to the processing cassette since the imaging plate 304 is sensitive to ambient energy, for example, bright ambient light. In particular, imaging plate 304 will begin to degrade from ambient light within two minutes of exposure to some ambient energies. In this aspect, in some embodiments, imaging plate 304 may be placed in a film bag or other similar type of “portable” darkroom which will protect imaging plate 304 from ambient energies while it is being transferred from carrier 104 to the processing cassette. It is further recognized that the sensitivity of imaging plate 304 to low level (subdued) light is significantly lower than for high energy x-rays. In particular, no noticeable difference has been observed between equine intraoral digital images processed with imaging plate 304 exposed to subdued light for the limited time required to transfer imaging plate 304 to the processing cassette as compared to images processed from imaging plate 304 transferred to the processing cassette using the film bag or portable darkroom. In particular, in subdued light, imaging plate 304 does not degrade for at least about two minutes while transfer typically takes only about ten seconds. Accordingly, it is contemplated that in other embodiments, image plate 304 may be transferred from carrier 104 to the processing cassette under subdued light conditions without harming the image stored in imaging plate 304.

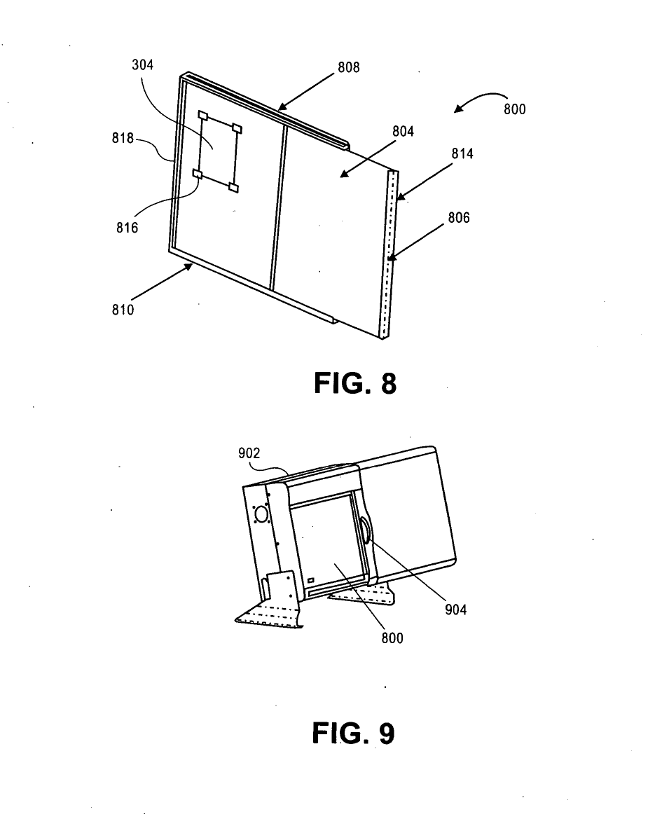

FIG. 8 is a diagram of a partially opened processing cassette. Processing cassette 800 may be similar in some respects to, for example, a 10″×12″ processing cassette commercially available from iCRco, Inc. of Torrance, Calif. However, the commercially available cassette includes a non-removable 10″×12″ imaging plate. Thus, for use in embodiments of the invention existing, commercially available cassettes must be modified to remove the existing fixed imaging plates and add a securing mechanism consistent with the size of the removable imaging plate to be used to hold the imaging plate in position. In one embodiment, processing cassette 800 includes housing 810 to enclose imaging plate 304. In this aspect, housing 810 is constructed primarily from carbon fiber, aluminum, Delrin plastic and similar materials that protect imaging plate 304 from ambient energy sources.

Processing cassette 800 may include sliding cover 804 or a similar mechanism to provide access to an internal cavity of processing cassette 800 where imaging plate 304 is positioned. In another embodiment, other exposure mechanisms may be employed to control access to imaging plate 304 stored within housing 810. In one embodiment, processing cassette 800 meets national emergency medicine association (NEMA) standards for size of x-ray cassettes.

Since conventional imaging plates are much larger than imaging plate 106 used for the intraoral x-ray techniques described herein, the internal cavity of commercial cassettes are too large to hold imaging plate 106 in place. In this aspect, the internal cavity of processing cassette 800 includes securing mechanisms 816 to secure edges of imaging plate 304 placed within the internal cavity. In some embodiments, securing mechanisms 816 may be secured to a bottom surface of the internal cavity at various points along a region of the surface defined by dimensions of imaging plate 304. In this aspect, securing mechanisms 816 may be L-shaped or T-shaped structures such that when an end of the structure is attached to the surface of the internal cavity, channels are formed between a surface of securing mechanisms 816 and the internal cavity for receiving edges of imaging plate 304 as shown. In this embodiment, suitable materials for securing mechanisms 816 may be any imaging agnostic material. An “imaging agnostic material” as used herein generally refers to any material which does not interfere with scanning of the imaging plate, by for example, preventing the scanner laser from exciting storage phosphors on the imaging plate or reading of the imaging plate by the scanner. Such materials, may include, for example, acrylic. Other embodiments, may use, for example, an aluminum securing mechanism 816 selected to minimize the overlap with the imaging plate within the scanning plane. Securing mechanisms 816 may be attached to the surface of the internal structure by, for example, screws, adhesives or any other suitable attachment mechanism. Alternatively, securing mechanisms 816 may be of any material and have any structure suitable for holding any number of imaging plates 304 having varying sizes within processing cassette 800 without interfering with scanning of the imaging plate 304. For example, securing mechanisms 816 may be a tape material, such as Scotch® tape which is adhered to the surface of internal cavity and imaging plate 304 to hold imaging plate 304 in position.

In one embodiment, at either end of processing cassette 800 there may be a set of steel strips 814, 818. Steel strips 814, 818 may be magnetized. In another embodiment, other magnetizable materials may be used. Steel strip 818 may be attached to the housing. Steel strip 814 may be attached to sliding cover 804. Cover 804 may slide in a track defined by housing 810 or move in relation to housing 810 by hinge or similar mechanism. In one embodiment, cover may be removed from processing cassette 800. Cover 804 may be constructed of carbon fiber, aluminum, Delrin plastic or similar materials. Steel strips 814, 818 may be used to move or hold cover 804 in relation to housing 810 in order to open and close processing cassette 800.

Steel strips 814, 818 may include notch 806. Notch 806 may be used to position processing cassette 800 in relation to a scanner. Magnetized protrusions may be attached to the scanner to interact with the notches 806 to allow manipulation of the cassette to open and close the cassette. In another embodiment, physical interconnections between processing cassette and scanner 902 (see FIG. 9) may be used to move cover 804 in relation to housing 810.

In one embodiment, processing cassette 800 may include an attachment mechanism 808 along an edge or side of processing cassette 800. Attachment mechanism 808 may be a clip, groove to fit a complementary tongue, hook or similar connection mechanism.

In one embodiment, processing cassette 800 encloses imaging plate 304. Imaging plate 304 may be exposed to a scanner or for other purposes by movement of housing 810 in relation to cover 804. Cover 804 may be form fit into a set of tracks in the housing allowing easy movement between an open and closed position.

FIG. 9 is a diagram of a scanner with a processing cassette positioned therein. FIG. 9 illustrates the final placement of processing cassette 800 in scanner 902. In one embodiment, processing cassette 800 is laid flat against a surface of scanner 902. Scanner 902 may be that which is commercially available from iCRco, Inc., of Torrance, Calif., under the model numbers iCR2600, iCRVet, iCR1000, iCR3600 and iCR3600SF. In this aspect, processing cassette 800 may be coupled in a removable manner to scanner 902. The coupling mechanism may be a complementary hook, slot, groove, clip or similar system. In another embodiment, processing cassette 800 is form fit or set in a track in scanner 902. Scanner 902 may include a sliding or hinged door 904 to a panel (not shown) that may completely enclose processing cassette 800.

In one embodiment, scanner 902 may move processing cassette 800 in a lateral or horizontal direction across the surface of scanner 902. Scanner 902 may include a scanning head such as a photo multiplier or charged coupled device (CCD) to capture the image of imaging plate 304 as it is moves past the head. A set of magnets in scanner 902 may exert force on steel strips 814, 818 to move housing 810 while holding cover 804 in place to expose imaging plate 304 to the scanner head. In another embodiment, imaging plate 304 may be attached to cover 804 and cover 804 moved past scanner 902. In a further embodiment, alternative movement mechanisms may be used to move housing 810 and cover 804. The movement mechanism may physically grip either cover 804 or housing 810 to move each component in relation to the other and to the scan head. Other movement mechanisms may include rollers, moving tracks, belts and similar mechanisms. Scanner 902 may also include a laser, set of light emitting diodes (LED) or similar energy source to erase imaging plate 304. Once imaging plate 304 is erased, it may be removed from processing cassette 800 and placed back into carrier 104 for reuse.

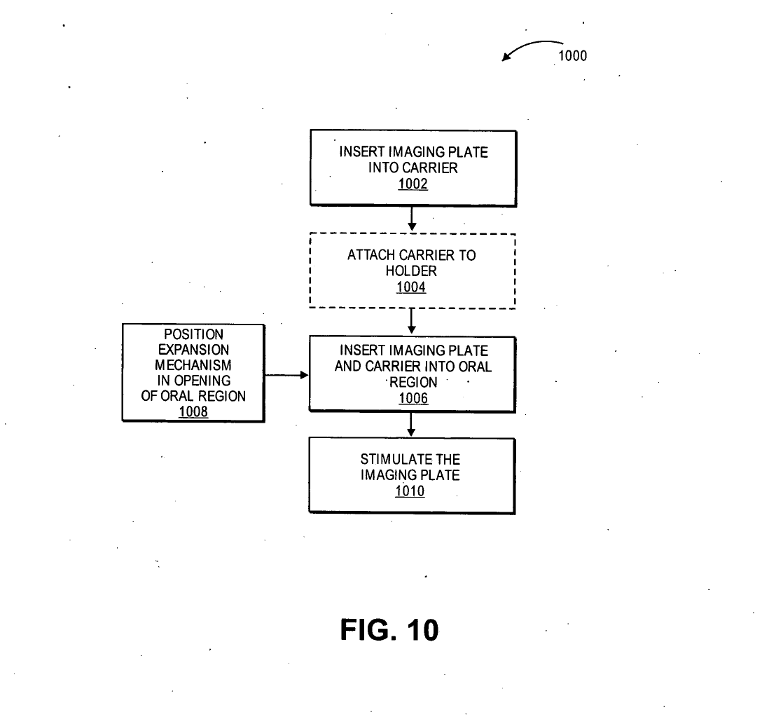

FIG. 10 is a flow chart of an imaging operation of a system for intraoral imaging 1000. In this embodiment, imaging plate 304, for example a phosphorescent plate, is inserted into housing 106 of carrier 104 (block 1002). In some embodiments, carrier 104 may optionally be attached to holder 502 to facilitate insertion and positioning of carrier 104 with imaging plate 304 inside within oral region 108 of the equine 102 (block 1004). Carrier 104 with imaging plate 304 inside, with or without holder 502, is then inserted into oral region 108 of equine 102 (block 1006). In addition, prior to insertion, expansion mechanism 602 may be inserted into an opening of oral region 108 and expanded to dilate the opening and hold it open during insertion of carrier 104 and subsequent examination of oral region 108 (block 1008). Once carrier 104 is in position, imaging plate 304 is stimulated with, for example, radiation from an x-ray generator (block 1010). It is contemplated that any conventional x-ray generator that can be properly positioned around the oral region of the animal may be used.

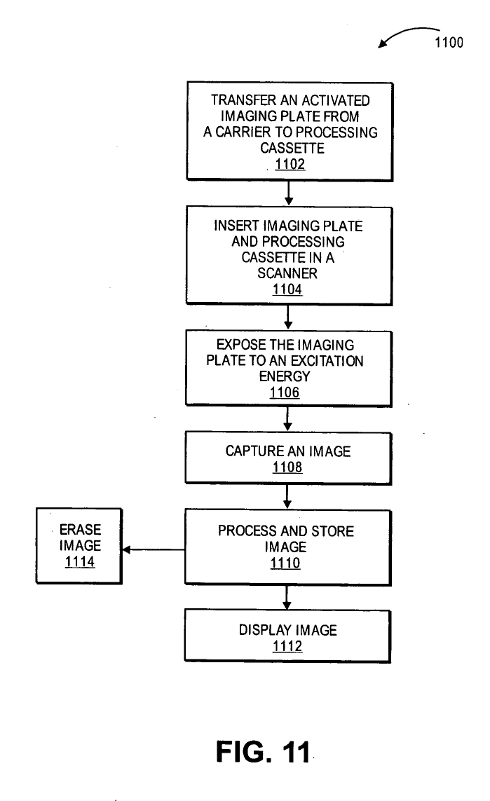

FIG. 11 is a flow chart of a processing operation of a system for intraoral imaging 1100. In this embodiment, an exposed or activated imaging plate 304 is removed from housing 106 of carrier 104 and transferred to processing cassette 800 (block 1102). Imaging plate 304 is positioned within processing cassette 80 as described in reference to FIG. 8. Processing cassette 800 with imaging plate 304 inside is inserted into scanner 902. Scanner 902 exposes imaging plate 304 to an excitation energy and captures an image from imaging plate 304 (blocks 1106, 1108). This image may then be processed and stored by a computing device (block 1110). Once the image is captured by scanner 902 and processed by the computing device, imaging plate 304 may be erased and reinserted into carrier 104 for reuse (block 1114). In some embodiments, the computing device is a high level computer, such as a Pentium IV computer, Windows XP or Windows 2000 having a 2 GB RAM and 80 GB HDD or higher. Still further, the computing device includes software which is coherent with scanner 902, such as software commercially available from iCRco, Inc. under the model number Xscan32. It is further contemplated that the computing device may include software to create a compact disk (CD) or DVD of the image. In some embodiments, the image may be displayed on a monitor connected to the computing device (block 1112). A suitable monitor may be any commercially available monitor having a high resolution, for example, a commercial grade high resolution color liquid-crystal display (LCD) (2-Megapixel) with a high contrast ratio (800:1 to 1000:1) or a medical grade monochrome monitor.

In some embodiments, a deep cell battery and pure sine-wave inverter may be provided to provide power to the computer and scanner 902 on site. In other embodiments, power may be drawn from the site where imaging is performed for example, in a barn.

It should be appreciated that reference throughout this specification to “one embodiment” or “an embodiment” means that a particular feature, structure or characteristic described in connection with the embodiment is included in at least one embodiment of the present invention. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the invention.

In the foregoing specification, the invention has been described with reference to specific embodiments thereof. It will, however, be evident that various modifications and changes can be made thereto without departing from the broader spirit and scope of the invention as set forth in the appended claims. The specification and drawings are, accordingly, to be regarded in an illustrative rather than a restrictive sense.

What is claimed is:

1. A method comprising:
positioning a reusable cassette comprising a flexible housing and a photostimulable plate within an oral region of a non-human animal;
stimulating the photostimulable plate; and
scanning the activated photostimulable plate.

2. The method of claim 1, further comprising:
transferring the photostimulable plate from the reusable cassette to a processing cassette.

3. The method of claim 1, further comprising:
exposing the plate to an excitation energy; and
capturing a digital image obtained from the excited photostimulable plate.

4. The method of claim 1, further comprising:
erasing an image from the activated photostimulable plate.

5. The method of claim 1, further comprising:
positioning the reusable cassette within the oral cavity with a cassette holder.

6. The method of claim 1, wherein the reusable cassette has a rectangular shape comprising dimensions greater than 2¼ inches by 3 inches.

7. The method of claim 1, wherein the non-human animal is an equine.

8. A system comprising:
a phosphorescent plate;
a flexible reusable carrier dimensioned to hold the phosphorescent plate and reside within an oral cavity of a non-human animal;
a processing cassette to removably retain the phosphorescent plate; and
a scanner to expose the phosphorescent plate within the processing cassette to an excitation energy and capture an image revealed by the excitation.

9. The system of claim 8, further comprising:
a display mechanism for displaying an image obtained by the scanner.

10. The system of claim 8, wherein the processing cassette further comprises:
an imaging agnostic securing mechanism to hold the phosphorescent plate in position.

11. The system of claim 8, further comprising an expansion mechanism for expanding an opening to the oral cavity.

12. The system of claim 11, wherein the expansion mechanism is a speculum.

13. The system of claim 8, further comprising:
a holder for positioning the flexible reusable carrier in the oral cavity.
